(19) United States
(12) Patent Application Publication
(10) Pub. No.: US 2007/0173733 A1
(43) Pub. Date: Jul. 26, 2007

(54) Detection of and Interaction Using Mental States

(75) Inventors: Tan Thi Thai Le, Pyrmont, New South Wales (AU); Nam Hoai Do, Pyrmont, New South Wales (AU); Marco Kenneth Della Torre, Pyrmont, New South Wales (AU); William Andrew King, Pyrmont, New South Wales (AU); Hai Ha Pham, Pyrmont, New South Wales (AU); Emir Delic, Pyrmont, New South Wales (AU); Johnson Thie, Pyrmont, New South Wales (AU); Wing Hong Siu, Pyrmont, New South Wales (AU)

Correspondence Address: FISH & RICHARDSON P.C., PO BOX 1022, MINNEAPOLIS, MN, 55440-1022, US

(73) Assignee: EMOTIV SYSTEMS PTY LTD, New South Wales (AU)

(21) Appl. No.: 11/531,265

(22) Filed: Sep. 12, 2006

Related U.S. Application Data
(60) Provisional application No. 60/716,657, filed on Sep. 12, 2005.

(51) Int. Cl. A61B 5/04

(52) U.S. Cl. 600/544

(57) ABSTRACT

A method of detecting a mental state includes receiving, in a processor, bio-signals of a subject from one or more bio-signal detectors, and determining in the processor whether the bio-signals represent the presence of a particular mental state in the subject. A method of using the detected mental state includes receiving, in a processor, a signal representing whether a mental state is present in the subject. The mental state can be a non-deliberative mental state, such as an emotion, preference or sensation. A processor can configured perform the methods, and a computer program product, tangibly stored on machine readable medium can have instructions operable to cause a processor to perform the methods.

### CROSS-REFERENCE TO RELATED APPLICATIONS

This application claims priority to U.S. Application Ser. No. 60/716,657, filed on Sep. 12, 2005, which is incorporated by reference.

### BACKGROUND

The present invention relates generally to the detection of mental states, particularly non-deliberative mental states, and interaction with machines using those mental states.

Interactions between humans and machines are usually restricted to the use of cumbersome input devices such as keyboards, joy sticks or other manually operable devices. Use of such interfaces limit the ability of a user to provide only premeditated and conscious commands.

A number of input devices have been developed to assist disabled persons in providing such premeditated and conscious commands. Some of these input devices detect eyeball movement or are voice activated to minimize the physical movement required by a user in order to operate these devices. Nevertheless, such input devices must be consciously controlled and operated by a user. However, most human actions are driven by things that humans are not aware of or do not consciously control, namely by the non-conscious mind. Non-consciously controlled communication exists only in communication between humans, and is frequently referred to as “intuition”.

### SUMMARY

It would be desirable to provide a manner of facilitating non-consciously controlled communication between human users and machines, such as electronic entertainment platforms or other interactive entities, in order to improve the interaction experience for a user. It would also be desirable to provide a means of interaction of users with one more interactive entities that is adaptable to suit a number of applications, without requiring the use of significant data processing resources. It would also be desirable to provide a method of interaction between one or more users with one or more interactive entities that ameliorates or overcomes one or more disadvantages of known interaction systems. It would moreover be desirable to provide technology that simplifies human-machine interactions. It would be desirable for this technology to be robust and powerful, and to use natural unconscious human interaction techniques so that the human-machine interaction is as natural as possible for the human user.

In one aspect, the invention is directed to a method of detecting a mental state. The method includes receiving, in a processor, bio-signals of a subject from one or more bio-signal detectors, and determining in the processor whether the bio-signals represent the presence of a particular mental state in the subject.

Implementations of the invention can include one or more of the following features. The particular mental state can be a non-deliberative mental state, such as an emotion, preference, sensation, physiological state, or condition. A signal can be generated from the processor representing whether the particular mental state is present. The bio-signals may include electroencephalograph (EEG) signals. The bio-signals may be transformed into a different representation, values for one or more features of the different representation can be determined, and the values compared to a mental state signature. Determining the presence of a non-deliberative mental state may be performed substantially without calibration of the mental state signature. The receiving and determining may occur in substantially real time.

In another aspect, the invention is directed to a method of using a detected mental state. The method includes receiving, in a processor, a signal representing whether a mental state is present in a subject.

Implementations of the invention can include one or more of the following features. The particular mental state may be a non-deliberative mental state, such as an emotion, preference, sensation, physiological state, or condition. The signal may be stored, or an action may be selected to modify an environment based on the signal. Data may be stored representing a target emotion, an alteration to an environmental variable that is expected to alter an emotional response of a subject toward the target emotion may be determined by the processor, and the alteration of the environmental variable may be caused. Whether the target emotion has been evoked may be determined based on signals representing whether the emotion is present in the subject. Weightings representing an effectiveness of the environmental variable in evoking the target emotion may be stored and the weightings may be used in determining the alteration. The weightings may be updated with a learning agent based on the signals representing whether the emotion is present. The environmental variables may occur in a physical or virtual environment.

In another aspect, the invention is directed to a computer program product, tangibly stored on machine readable medium, the product comprising instructions operable to cause a processor to perform a method described above. In another aspect, the invention is directed to a system having a processor configured perform the method described above.

In another aspect, the invention is directed to a method of detecting and using a mental state. The method includes detecting bio-signals of a subject with one or more bio-signal detectors, directing the bio-signals to a first processor, determining in the first processor whether the bio-signals represent the presence of a particular mental state in the subject, generating a signal from the first processor representing whether the particular mental state is present, receiving the signal at a second processor, and storing the signal or modifying an environment based on the signal.

In another aspect, the invention is directed to an apparatus comprising one or more bio-signal detectors, a first processor configured to bio-signals from the one or more bio-signal detectors, determine whether the bio-signals indicate the presence of a particular mental state in a subject, and generate a signal representing whether the particular mental state is present, and a second processor configured to receive the signal and store the signal or modify an environment based on the signal.

In another aspect, the invention is directed to a method of interaction of a user with an environment. The method includes detecting and classifying the presence of a predetermined mental state in response to one or more biosignals from the user, selecting one or more environmental variables that affect an emotional response of the user, and performing one or more actions to alter the selected environmental variables and thereby alter the emotional response of a user.

The details of one or more embodiments of the invention are set forth in the accompanying drawings and the description below. Other features, objects, and advantages of the invention will be apparent from the description and drawings, and from the claims.

### DRAWINGS

FIG. 1 is a schematic diagram illustrating the interaction of a system for detecting and classifying mental states, such as non-deliberative mental states, for example emotions, with a system that uses the detected mental states, and a subject.

FIG. 1A is a schematic diagram of an apparatus for detecting and classifying mental states, such as non-deliberative mental states, such as emotions.

FIGS. 1B-1D are variants of the apparatus shown in FIG. 1A.

FIG. 2 is the schematic diagram illustrating the position of bio-signal detectors in the form of scalp electrodes forming part of a headset used in the apparatus shown in FIG. 1.

FIGS. 3 and 4 are flow charts illustrating the broad functional steps performed during detection and classification of mental states by the apparatus shown in FIG. 1; and

FIG. 5 is a graphical representation of bio-signals processed by the apparatus of FIG. 1 and the transformation of those bio-signals.

FIG. 6 is a schematic diagram of a platform for using the detected emotions to control environmental variables.

FIG. 7 is a flow chart illustrating the high level functionality of the apparatus and platform shown in FIG. 1 when in use.

FIGS. 8 and 9 are two variants of the platform shown in FIG. 4.

Like reference symbols in the various drawings indicate like elements.

### DESCRIPTION

The present invention relates generally to communication from users to machines. In particular, a mental state of a subject can be detected and classified, and a signal to represent this mental state can be generated and directed to a machine. The present invention also relates generally to a method of interaction using non-consciously controlled communication by one or more users with an interactive environment controlled by a machine. The invention is suitable for use in electronic entertainment platform or other platforms in which users interact in real time, and it will be convenient to describe the invention in relation to that exemplary but non limiting application.

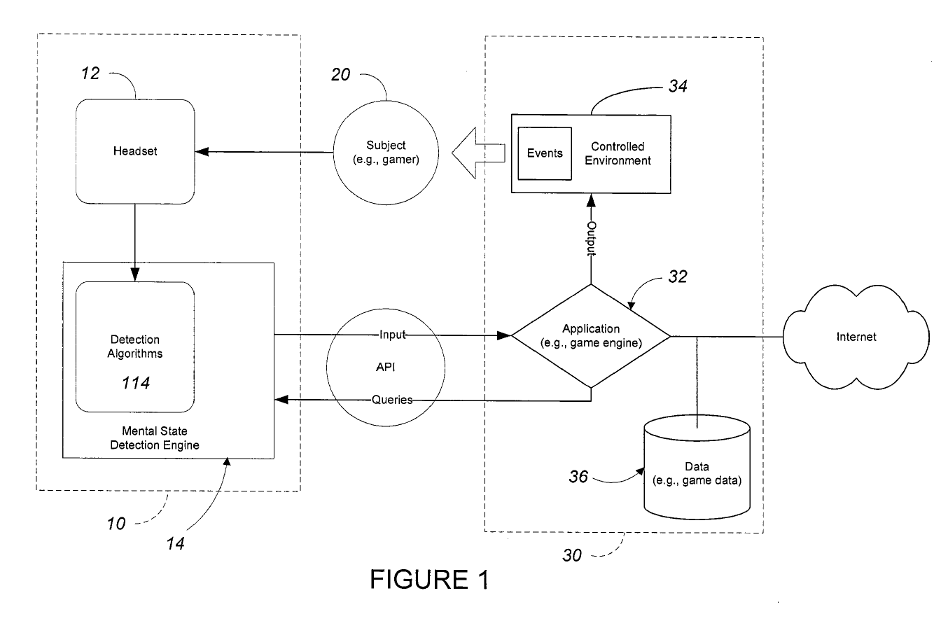

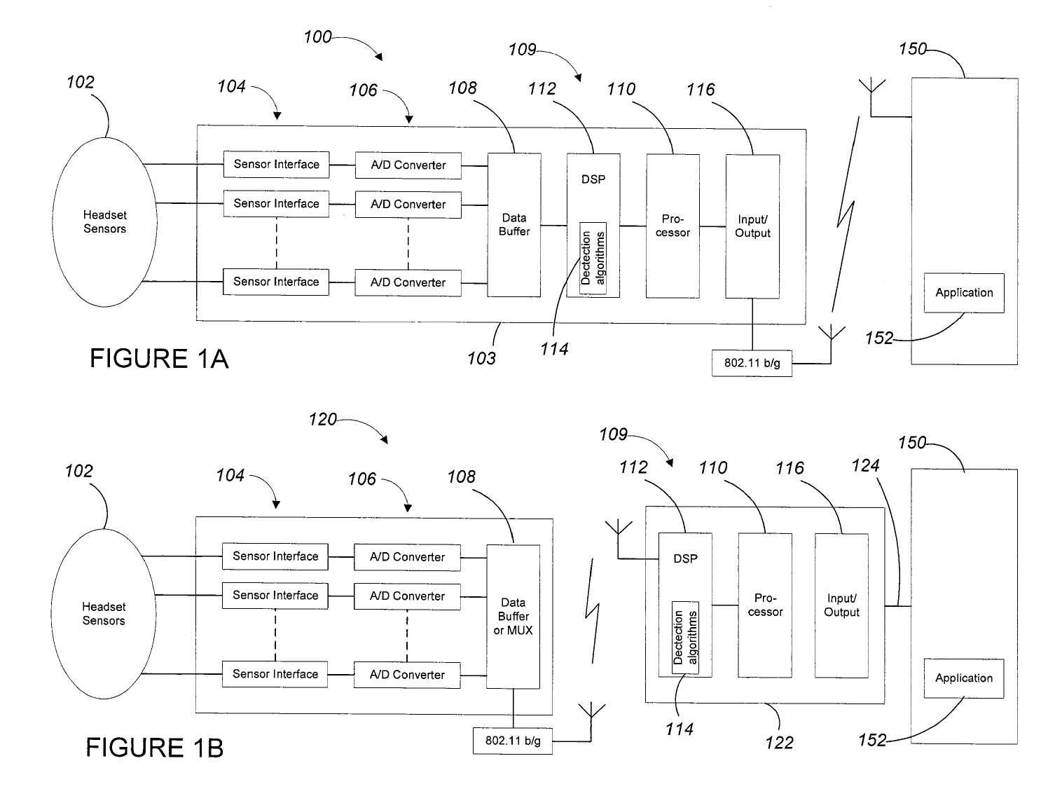

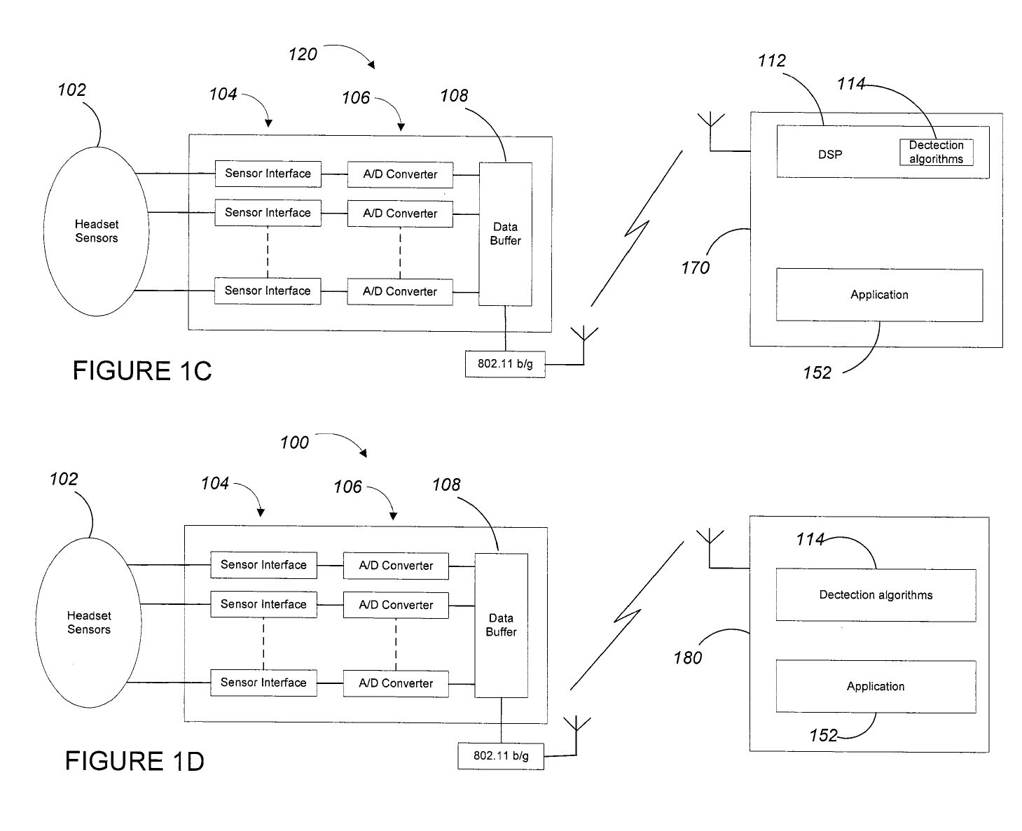

Turning now to FIG. 1, there is shown a system 10 for detecting and classifying deliberative or non-deliberative mental states of a subject and generating signals to represent these mental states. In general, non-deliberative mental states are mental states which lack the subjective quality of a volitional act. These non-deliberative mental states are sometime called the non-conscious mind, but it should be understood that in this context non-conscious refers to not consciously selected; non-deliberative mental states can be (although not all necessarily are) consciously experienced. In contrast, deliberative mental states occur when a subject consciously focuses on a task, image or willed experience.

There are several categories of non-deliberative mental states, including emotions, preference, sensations, physiological states, and conditions, that can be detected by the system 10. “Emotions” include excitement, happiness, fear, sadness, boredom, and other emotions. “Preference” generally manifests as an inclination toward or away from (e.g., liking or disliking) something observed. “Sensations” include thirst, pain, and other physical sensations, and may be accompanied by a corresponding urge to relieve or enhance the sensation. “Physiological states” refer to brain states that substantially directly control body physiology, such as heart rate, body temperature, and sweatiness. “Conditions” refer to brain states that are causes, symptoms or side-effects of a bodily condition, yet are not conventionally associated with sensations or physiological states. An epileptic fit is one example of a condition. The way that the brain processes visual information in the occipital lobe when a person has glaucoma is another example of a condition. Of course, it should be understood that some non-deliberative mental states might be classified into more than one of these categories, or might not fit well into any of these categories.

The system 10 includes two main components, a neuro-physiological signal acquisition device 12 that is worn or otherwise carried by a subject 20, and a mental state detection engine 14. In brief, the neuro-physiological signal acquisition device 12 detects bio-signals from the subject 20, and the mental state detection engine 14 implements one or more detection algorithms 114 that convert these bio-signals into signals representing the presence (and optionally intensity) of particular mental states in the subject. The mental state detection engine 14 includes at least one processor, which can be a general-purpose digital processor programmed with software instructions, or a specialized processor, e.g., an ASIC, that perform the detection algorithms 114. It should be understood that, particularly in the case of a software implementation, the mental state detection engine 14 could be a distributed system operating on multiple computers.

In operation, the mental state detection engine can detect mental states practically in real time, e.g., less than a 50 millisecond latency is expected for non-deliberative mental states. This can enable detection of the mental state with sufficient speed for person-to-person interaction, e.g., with avatars in a virtual environment being modified based on the detected mental state, without frustrating delays. Detection of deliberative mental states may be slightly slower, e.g., with less than a couple hundred milliseconds, but is sufficiently fast to avoid frustration of the user in human-machine interaction.

The detection algorithms 114 are described in more detail below, and in co-pending U.S. patent application Ser. No. 11/225,835, filed Sep. 12, 2005 and patent application Ser. No. 11/531,238, filed Sep. 12, 2006, each of which is incorporated by reference.

The mental state detection engine 14 is coupled by an interface, such as an application programming interface (API), to a system 30 that uses the signals representing mental states. The system 30 includes an application engine 32 that can generate queries to the system 10 requesting data on the mental state of the subject 20, and receive input signals that represent the mental state of the subject, and use these signals. Thus, the results of the mental state detection algorithms are directed to they system 30 as input signals representative of the predetermined non-deliberative mental state. Optionally, the system 30 can control an environment 34 to which the subject is exposed, and can use the signals that represent the mental state of the subject can to determine events to perform that will modify the environment 34. For example, the system 30 can store data representing a target emotion, and can control the environment 34 to evoke the target emotion. Alternatively, the system can be used primarily for data collection, and can store and display information concerning the mental state of the subject to a user (who might not be the subject) in a human-readable format. The system 30 can include a local data store 36 coupled to the engine 32, and can also be coupled to a network, e.g., the Internet. The engine 32 can include at least one processor, which can be a general-purpose digital processor programmed with software instructions, or a specialized processor, e.g., an ASIC. In addition, it should be understood that the system 30 could be a distributed system operating on multiple computers.

The neuro-physiological signal acquisition device 12 includes bio-signal detectors capable of detecting various bio-signals from a subject, particularly electrical signals produced by the body, such as electroencephalograph (EEG) signals, electrooculargraph (EOG) signals, electomyograph (EMG) signals, and the like. It should be noted, however, that the EEG signals measured and used by the system 10 can include signals outside the frequency range, e.g., 0.3-80 Hz, that is customarily recorded for EEG. It is generally contemplated that the system 10 is capable of detection of mental states (both deliberative and non-deliberative) using solely electrical signals, particularly EEG signals, from the subject, and without direct measurement of other physiological processes, such as heart rate, blood pressure, respiration or galvanic skin response, as would be obtained by a heart rate monitor, blood pressure monitor, and the like. In addition, the mental states that can be detected and classified are more specific than the gross correlation of brain activity of a subject, e.g., as being awake or in a type of sleep (such as REM or a stage of non-REM sleep), conventionally measured using EEG signals. For example, specific emotions, such as excitement, or specific willed tasks, such as a command to push or pull an object, can be detected.

In an exemplary embodiment, the neuro-physiological signal acquisition device includes a headset that fits on the head of the subject 20. The headset includes a series of scalp electrodes for capturing EEG signals from a subject or user. These scalp electrodes may directly contact the scalp or alternatively may be of a non-contact type that do not require direct placement on the scalp. Unlike systems that provide high-resolution 3-D brain scans, e.g., MRI or CAT scans, the headset is generally portable and non-constraining.

The electrical fluctuations detected over the scalp by the series of scalp electrodes are attributed largely to the activity of brain tissue located at or near the skull. The source is the electrical activity of the cerebral cortex, a significant portion of which lies on the outer surface of the brain below the scalp. The scalp electrodes pick up electrical signals naturally produced by the brain and make it possible to observe electrical impulses across the surface of the brain.

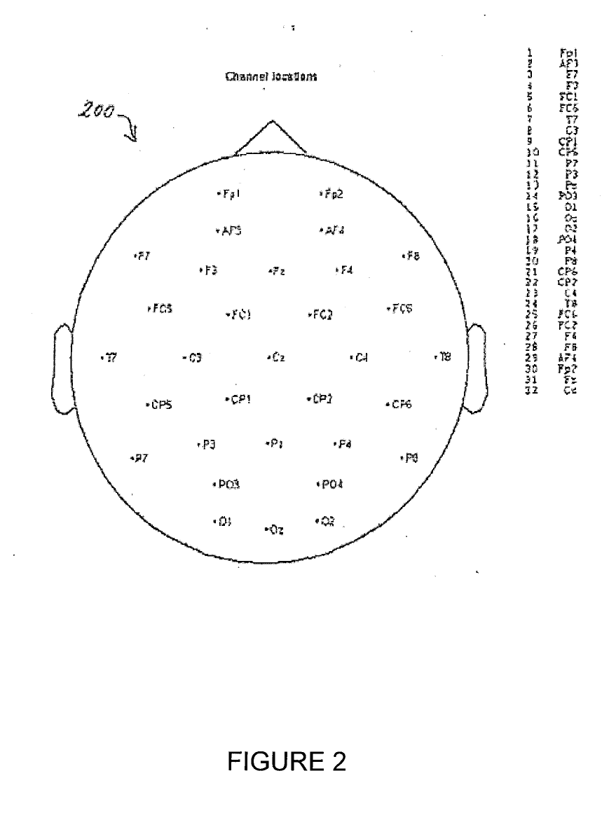

FIG. 2 illustrates one example of the positioning of the scalp electrodes forming part of the headset. The electrode placement shown in FIG. 2 is referred to as the “10-20” system and is based on the relationship between the location of an electrode and the underlying area of the cerebral cortex. Each point on the electrode placement system 200 indicates a possible scalp electrode position. Each side indicates a letter to identify the load and number or other letter to identify the hemisphere location. The letters F, T, C, P and 0 stand for Frontal, Temporal, Central, Parietal and Occipital. Even numbers refer to the right hemisphere and odd mbers refer to the left hemisphere. The letter Z refers to an electrode placed on the mid line. The mid-line is a line along the scalp on the sagittal plane originating at the nasion and ending at the inion at the back of the head. The “10” and “20” refer to percentages of the mid-line division. The mid-line is divided into 7 positions, namely, Nasion, Fpz, Fz, Cz, Pz, Oz and Inion, and the angular intervals between adjacent positions are 10%, 20%, 20%, 20%, 20% and 10% of the mid-line length respectively.

Although in this exemplary embodiment the headset includes thirty-two scalp electrodes, other embodiments could include a different number and different placement of the scalp electrodes. For example, the headset could include sixteen electrodes plus reference and ground.

Turning to FIG. 1A, there is shown an apparatus 100 that includes the system for detecting and classifying mental states, and an external device 150 that includes the system which uses the signals representing mental states. The apparatus 100 includes a headset 102 as described above, along with processing electronics 103 to detect and classify mental states of the subject from the signals from the headset 102.

Each of the signals detected by the headset 102 is fed through a sensory interface 104, which can include an amplifier to boost signal strength and a filter to remove noise, and then digitized by an analog-to-digital converter 106. Digitized samples of the signal captured by each of the scalp sensors are stored during operation of the apparatus 103 in a data buffer 108 for subsequent processing. The apparatus 100 further includes a processing system 109 which includes a digital signal processor (DSP) 112, a co-processor 110, and associated memory for storing a series of instructions, otherwise known as a computer program or a computer control logic, to cause the processing system 109 to perform desired functional steps. The co-processor 110 is connected through an input/output interface 116 to a transmission device 118, such as a wireless 2.4 GHz device, a WiFi or Bluetooth device, or an 802.11b/g device. The transmission device 118 connects the apparatus 100 to the external device 150.

Notably, the memory includes a series of instructions defining at least one algorithm 114 that will be performed by the digital signal processor 112 for detecting and classifying a predetermined non-deliberative mental state. In general, the DSP 112 performs preprocessing of the digital signals to reduce noise, transforms the signal to “unfold” it from the particular shape of the subject's cortex, and performs the emotion detection algorithm on the transformed signal. The emotion detection algorithm can operate as a neural network that adapts to the particular subject for classification and calibration purposes. In addition to the emotion detection algorithms, the DSP can also store the detection algorithms for deliberative mental states and for facial expressions, such as eye blinks, winks, smiles, and the like. Detection of facial expression is described in U.S. patent application Ser. No. 11/225,598, filed Sep. 12, 2005, and in U.S. patent application Ser. No. 11/531,117, filed Sep. 12, 2006, each of which is incorporated by reference.

The co-processor 110 performs as the device side of the application programming interface (API), and runs, among other functions, a communication protocol stack, such as a wireless communication protocol, to operate the transmission device 118. In particular, the co-processor 110 processes and prioritizes queries received from the external device 150, such as a queries as to the presence or strength of particular non-deliberative mental states, such as emotions, in the subject. The co-processor 110 converts a particular query into an electronic command to the DSP 112, and converts data received from the DSP 112 into a response to the external device 150.

In this embodiment, the mental state detection engine is implemented in software and the series of instructions is stored in the memory of the processing system 109. The series of instructions causes the processing system 109 to perform functions of the invention as described herein. In other embodiments, the mental state detection engine can be implemented primarily in hardware using, for example, hardware components such as an Application Specific Integrated Circuit (ASIC), or using a combination of both software and hardware.

The external device 150 is a machine with a processor, such as a general purpose computer or a game console, that will use signals representing the presence or absence of a predetermined non-deliberative mental state, such as a type of emotion. If the external device is a general purpose computer, then typically it will run one or more applications 152 that act as the engine to generate queries to the apparatus 100 requesting data on the mental state of the subject, to receive input signals that represent the mental state of the subject. The application 152 can also respond to the data representing the mental state of the user by modifying an environment, e.g., a real environment or a virtual environment. Thus, the mental state of the user can used as a control input for a gaming system, or another application (including a simulator or other interactive environment).

The system that receives and responds to the signals representing mental states can be implemented in software and the series of instructions can be stored in a memory of the device 150. In other embodiments, the system that receives and responds to the signals representing mental states can be implemented primarily in hardware using, for example, hardware components such as an Application Specific Integrated Circuit (ASIC), or using a combination of both software and hardware.

Other implementations of the apparatus 100 are possible. Instead of a digital signal processor, an FPGA (field programmable gate array) could be used. Rather than a separate digital signal processor and co-processor, the processing functions could be performed by a single processor. The buffer 108 could be eliminated or replaced by a multiplexer (MUX), and the data stored directly in the memory of the processing system. A MUX could be placed before the A/D converter stage so that only a single A/D converter is needed. The connection between the apparatus 100 and the platform 120 can be wired rather than wireless.

Although the mental state detection engine is shown in FIG. 1A as a single device, other implementations are possible. For example, as shown in FIG. 1B, the apparatus includes a head set assembly 120 that includes the head set, a MUX, A/D converter(s) 106 before or after the MUX, a wireless transmission device, a battery for power supply, and a microcontroller to control battery use, send data from the MUX or A/D converter to the wireless chip, and the like. The A/D converters 106, etc., can be located physically on the headset 102. The apparatus can also a separate processor unit 122 that includes a wireless receiver to receive data from the headset assembly, and the processing system, e.g., the DSP 112 and co-processor 110. The processor unit 122 can be connected to the external device 150 by a wired or wireless connection, such as a cable 124 that connects to a USB input of the external device 150. This implementation may be advantageous for providing a wireless headset while reducing the number of the parts attached to and the resulting weight of the headset.

As another example, as shown in FIG. 1C, a dedicated digital signal processor 112 is integrated directly into a device 170. The device 170 also includes a general purpose digital processor to run an application 114 or application-specific processor that will use the information on the non-deliberative mental state of the subject. In this case, the functions of the mental state detection engine are spread between the headset assembly 120 and the device 170 which runs the application 152. As yet another example, as shown in FIG. 1D, there is no dedicated DSP, and instead the mental state detection algorithms 114 are performed in a device 180, such as a general purpose computer, by the same processor that executes the application 152. This last embodiment is particularly suited for both the mental state detection algorithms 114 and the application 152 to be implemented with software and the series of instructions is stored in the memory of the device 180.

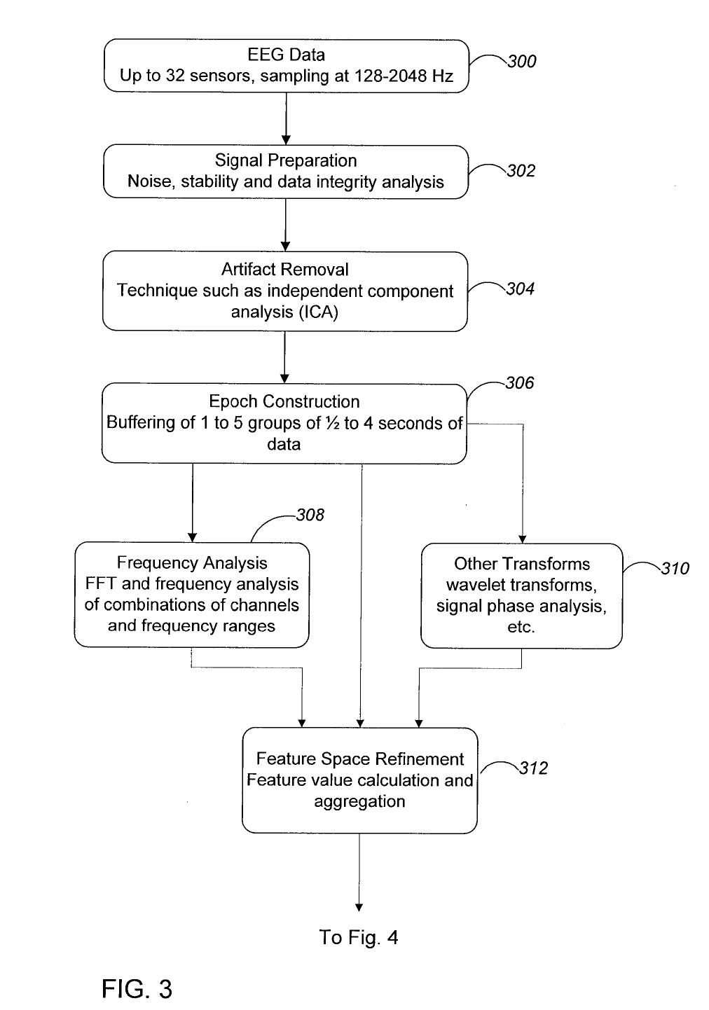

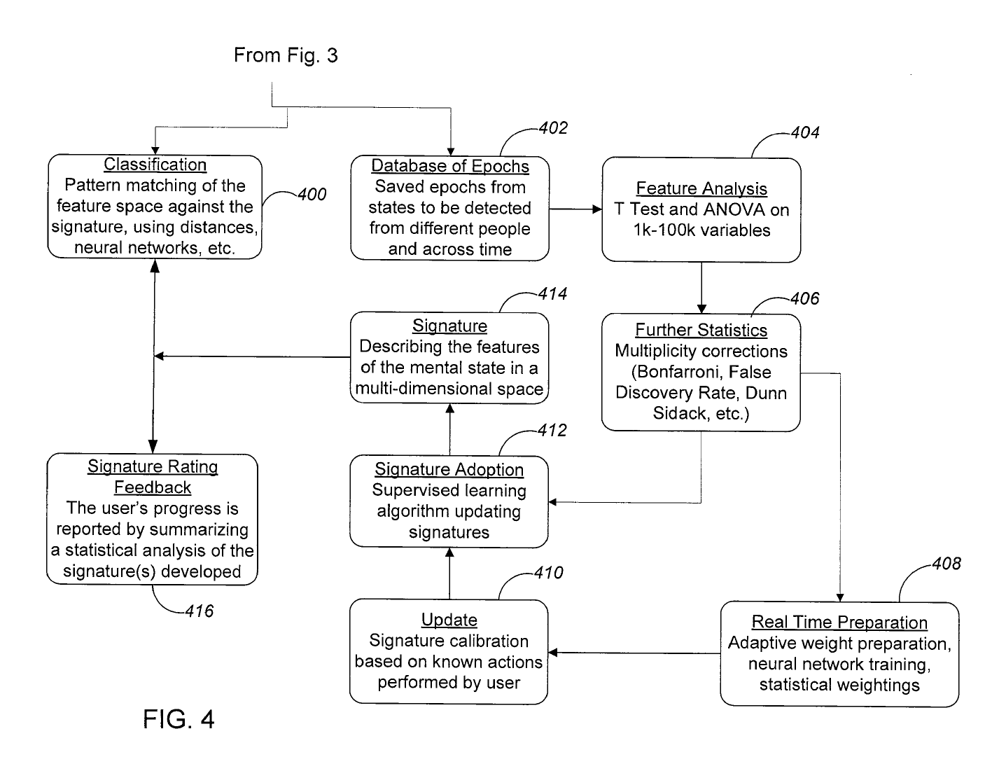

In operation, the headset 102, including scalp electrodes positioned according to the system 200, are placed on the head of a subject in order to detect EEG signals. FIG. 3 shows a series of steps carried out by the apparatus 100 during the capture of those EEG signals and subsequent data preparation operations carried out by the processing system 109.

At step 300, the EEG signals are captured and then digitised using the analogue to digital converters 106. The data samples are stored in the data buffer 108. The EEG signals detected by the headset 102 may have a range of characteristics, but for the purposes of illustration typical characteristics are as follows: Amplitude 10-4000 μV, Frequency Range 0.16-256 Hz and Sampling Rate 128-2048 Hz.

At step 302, the data samples are conditioned for subsequent analysis. Sources of possible noise that are desired to be eliminated from the data samples include external interference introduced in signal collection, storage and retrieval. For EEG signals, examples of external interference include power line signals at 50/60 Hz and high frequency noise originating from switching circuits residing in the EEG acquisition hardware. A typical operation carried out during this conditioning step is the removal of baselines via high pass filters. Additional checks are performed to ensure that data samples are not collected when a poor quality signal is detected from the headset 102. Signal quality information can be fed back to a user to help them to take corrective action.

An artefact removal step 304 is then carried out to remove signal interference. EEG signals consist, in this example, of measurements of the electrical potential at numerous locations on a user's scalp. These signals can be represented as a set of observations xₙ of some “signal sources” sm where nε[1:N], mε[1:M], n is channel index, N is number of channels, m is source index, M is number of sources. If there exists a set of transfer functions F and G that describe the relationship between sₘ and xₙ, one can then identify with a certain level of confidence which sources or components have a distinct impact on observation xₙ and their characteristics. Different techniques such as Independent Component Analysis (ICA) are applied by the apparatus 100 to find components with greatest impact on the amplitude of xₙ. These components often result from interference such as power line noise, signal drop outs, and muscle, eye blink, and eye movement artefacts.

The EEG signals are converted, in steps 306, 308 and 310, into different representations that facilitate the detection and classification of the mental state of a user of the headset 102.

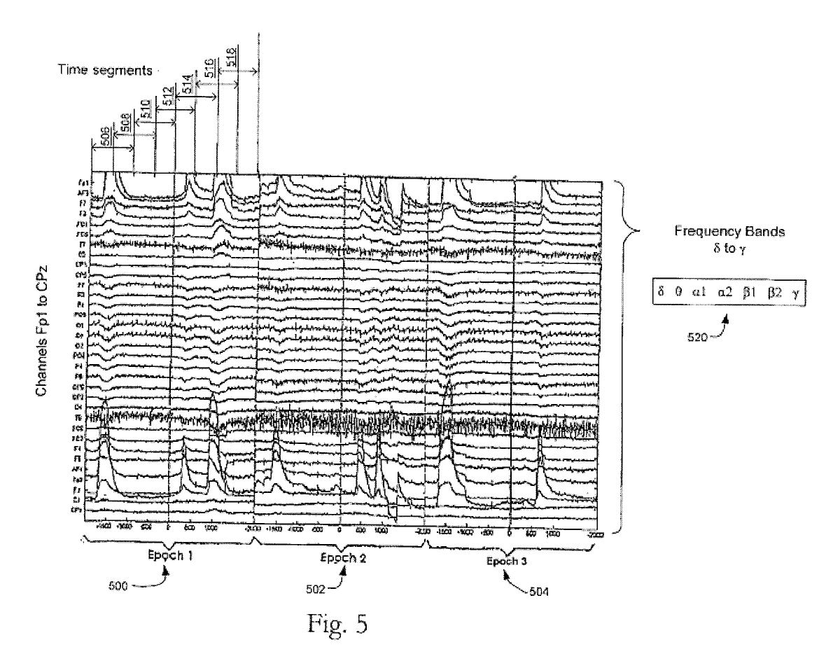

The data samples are firstly divided into equal length time segments within epochs, at step 306. While in the exemplary embodiment illustrated in FIG. 5 there are seven time segments of equal duration within the epoch, in another embodiment the number and length of the time segments may be altered. Furthermore, in another embodiment, time segments may not be of equal duration and may or may not overlap within an epoch. The length of each epoch can vary dynamically depending on events in the detection system such as artefact removal or signature updating. However, in general, an epoch is selected to be sufficiently long that a change in mental state, if one occurs, can be reliably detected. FIG. 5 is a graphical illustration of EEG signals detected from the 32 electrodes in the headset 102. Three epochs 500, 502 and 504 are shown each with 2 seconds before and 2 seconds after the onset of a change in the mental state of a user. In general, the baseline before the event is limited to 2 seconds whereas the portion after the event (EEG signal containing emotional response) varies, depending on the current emotion that is being detected.

The processing system 109 divides the epochs 500, 502 and 504 into time segments. In the example shown in FIG. 5, the epoch 500 is divided into 1 second long segments 506 to 518, each of which overlap by half a second. A 4 second long epoch would then yield 7 segments.

The processing system 109 then acts in steps 308 and 310 to transform the EEG signal into the different representations so that the value of one or more features of each EEG signal representation can be calculated and collated at step 312. For example, for each time segment and each channel, the EEG signal can be converted from the time domain (signal intensity as a function of time) into the frequency domain (signal intensity as a function of frequency). In an exemplary embodiment, the EEG signals are band-passed (during transform to frequency domain) with low and high cut-off frequencies of 0.16 and 256 Hz, respectively.

As another example, the EEG signal can be converted into a differential domain (marginal changes in signal intensity as a function of time) that approximates a first derivative. The frequency domain can also be converted into a differential domain (marginal changes in signal intensity as a function of frequency), although this may require comparison of frequency spectrums from different time segments.

In step 312 the value of one or more features of each EEG signal representation can be calculated (or collected from previous steps if the transform generated scalar values), and the various values assembled to provide a multi-dimensional representation of the mental state of the subject. In addition to values calculated from transformed representations of the EEG signal, some values could be calculated from the original EEG signals.

As an example of the calculation of the value of a feature, in the frequency domain, the aggregate signal power in each of a plurality of frequency bands can be calculated. In an exemplary embodiment described herein, seven frequency bands are used with the following frequency ranges: δ(2-4 Hz), θ(4-8 Hz), α1(8-10 Hz) α2(10 13 Hz), β1(13-20 Hz), β2(20-30 Hz) and γ(30-45). The signal power in each of these frequency bands is calculated. In addition, the signal power can be calculated for various combinations of channels or bands. For example, the total signal power for each spatial channel (each electrode) across all frequency bands could be determined, or the total signal power for a given frequency band across all channels could be determined.

In other embodiments of the invention, both the number of and ranges of the frequency bands may be different to the exemplary embodiment depending notably on the particular application or detection method employed. In addition, the frequency bands could overlap. Furthermore, features other than aggregate signal power, such as the real component, phase, peak frequency, or average frequency, could be calculated from the frequency domain representation for each frequency band.

In this exemplary embodiment, the signal representations are in the time, frequency and spatial domains. The multiple different representations can be denoted as xᵢⱼₖⁿ where n, i, j, k are epoch, channel, frequency band, and segment index, respectively. Typical values for these parameters are:

iε[1:32] 32 spatially distinguishable channels (referenced Fp₁ to CPz)

jε[1:7] 7 frequency distinguishable bands (referenced δ to γ)

The operations carried out in step 310-312 often produce a large number of state variables. For example, calculating correlation values for 2 four-second long epochs consisting of 32 channels, using 7 frequency bands gives more than 1 million state variables:
³²C₂x7²x7²=1190896

Since individual EEG signals and combinations of EEG signals from different sensors can be used, as well as wide range of features from a variety of different transform domains, the number of dimensions to be analysed by the processing system 109 is extremely large. This huge number of dimensions enables the processing system 109 to detect a wide range of mental states, since the entire or a significant portion of the cortex and a full range of features are considered in detecting and classifying a mental state.

Other common features to be calculated by the processing system 109 at step 312 include the signal power in each channel, the marginal changes of the power in each frequency band in each channel, the correlations/coherence between different channels, and the correlations between the marginal changes of the powers in each frequency band. The choice between these properties depends on the types of mental state that are desired to distinguish. In general, marginal properties are more important in case of short term emotional burst whereas in a long term mental state, other properties are more significant.

A variety of techniques can be used to transform the EEG signal into the different representations and to measure the value of the various features of the EEG signal representations. For example, traditional frequency decomposition techniques, such as Fast Fourier Transform (FFT) and band-pass filtering, can be carried out by the processing system 109 at step 308, whilst measures of signal coherence and correlation can be carried out at step 310 (in this later case, the coherence or correlation values can be collated in step 312 to become part of the multi-dimensional representation of the mental state). Assuming that the correlations/coherence is calculated between different channels, this could also be considered a domain, e.g., a spatial coherence/correlation domain (coherence/correlation as a function of electrode pairs). For example, in other embodiments, a wavelet transform, dynamical systems analysis or other linear or non-linear mathematical transform may be used in step 310.

The FFT is an efficient algorithm of the discrete Fourier transform which reduces the number of computations needed for N data points from 2N₂ to 2N log₂N. Passing a data channel in time domain through an FFT, will generate a description for that data segment in the complex frequency domain.

Coherence is a measure of the amount of association or coupling between two different time series. Thus, a coherence computation can be carried out between two channels a and b, in frequency band Cn, where the Fourier components of channels a and b of frequency fμ are xaμ and xbμ is:

Thus, a coherence computation can be carried out between two channels α and b, in frequency band ωₙ, where the Fourier components of channels α and b of frequency fᵤ are xₐᵤ and x_{bu} is:

$${C}_{ab {ω}_{n}} \frac{{\sum }_{{f}_{μ} ∈ {ω}_{n}} {x}_{a μ} {X}_{b μ}^{*}}{\sqrt{{\sum }_{{f}_{μ} ∈ {ω}_{n}} {x}_{a μ}^{2}} \sqrt{{\sum }_{{f}_{μ} ∈ {ϖ}_{n}} {x}_{b μ}^{2}}}$$

Correlation is an alternative to coherence to measure the amount of association or coupling between two different time series. For the same assumption as of coherence section above, a correlation r_{ab}, computation can be carried out between the signals of two channels xₐ(tᵢ) and x_{b}(tᵢ), is defined as,

$${r}_{ab} = \frac{\sum ( {x}_{ai} - \overline{{x}_{a}} ) ( {x}_{bi} - \overline{{x}_{b}} )}{\sqrt{{\sum }_{i} {( {x}_{ai} - \overline{{x}_{a}} )}^{2}} {\sum }_{j} {( {x}_{bj} - {x}_{b} )}^{2}}$$

where xₐᵢ and x_{bi} have already had common band-pass filtering 1010 applied to them.

FIG. 4 shows in the various data processing operations, preferably carried out in real-time, which are then carried out by the processing system 109. At step 400, the calculated values of one or more features of each signal representation are compared to one or more mental state signatures stored in the memory of the processing system 109 to classify the mental state of the user. Each mental state signature defines reference feature values that are indicative of a predetermined mental state.

A number of techniques can be used by the processing device 109 to match the pattern of the calculated feature values to the mental state signatures. A multi layer perceptron neural network can be used to classify whether a signal representation is indicative of a mental state corresponding to a stored signature. The processing system 109 can use a standard perceptron with n inputs, one or more hidden layers of m hidden nodes and an output layer with l output nodes. The number of output nodes is determined by how many independent mental states the processing system is trying to recognize. Alternately, the number of networks used may be varied according to the number of mental states being detected. The output vector of the neural network can be expressed as,
Y=F₂(W₂·F₁(W₁·X))
where W₁ is m by (n+1) weight matrix, W₂ is an l by (m+1) weight matrix (the additional column in the weight matrices allows for a bias term to be added) and X=(X₁,X₂, . . . Xₙ) is the input vector. F₁ and F₂ are the activation functions that act on the components of the column vectors separately to produce another column vector and Y is the output vector. The activation function determines how the node is activated by the inputs. The processing system 109 uses a sigmoid function. Other possibilities are a hyperbolic tangent function or even a linear function. The weight matrices can be determined either recursively or all at once.

Distance measures for determining similarity of an unknown sample set to a known one can be used as an alternative technique to the neural network. Distances such as the modified Mahalanobis distance, the standardised Euclidean distance and a projection distance, can be used to determine the similarity between the calculated feature values and the reference feature values defined by the various mental state signatures to thereby indicate how well a user's mental state reflects each of those signatures.

The mental state signatures and weights can be predefined. For example, for some mental states, signatures are sufficiently uniform across a human population that once a particular signature is developed (e.g., by deliberately evoking the mental state in test subjects and measuring the resulting signature), this signature can be loaded into the memory and used without calibration by a particular user. On the other hand, for some mental states, signatures are sufficiently non-uniform across the human population that predefined signatures cannot be used or can be used only with limited satisfaction by the subject. In such a case, signatures (and weights) can be generated by the apparatus 100, as discussed below, for the particular user (e.g., by requesting that the user make a willed effort for some result, and measuring the resulting signature). Of course, for some mental states the accuracy of a signature and/or weights that was predetermined from test subjects can be improved by calibration for a particular user. For example, to calibrate the subjective intensity of a non-deliberative mental state for a particular user, the user could be exposed to a stimulus that is expected to produce a particular mental state, the resulting bio-signals compared to a predefined signature. The user can be queried regarding the strength of the mental state, and the resulting feedback from the user applied to adjust the weights. Alternatively, calibration could be performed by a statistical analysis of the range of stored multi-dimensional representations. To calibrate a deliberative mental state, the user can be requested to make a willed effort for some result, and the multi-dimensional representation of the resulting mental state can be used to adjust the signature or weights.

The apparatus 100 can also be adapted to generate and update signatures indicative of a user's various mental states. At step 402, data samples of the multiple different representations of the EEG signals generated in steps 300 to 310 are saved by the processing system 109 in memory, preferably for all users of the apparatus 100. An evolving database of data samples is thus created which allows the processing device 109 to progressively improve the accuracy of mental state detection for one or more users of the apparatus 100.

At step 404, one or more statistical techniques are applied to determine how significant each of the features is in characterising different mental states. Different coordinates are given a rating based on how well they differentiate. The techniques implemented by the processing system 109 use a hypothesis testing procedure to highlight regions of the brain or brainwave frequencies from the EEG signals, which activate during different mental states. At a simplistic level, this approach typically involves determining whether some averaged (mean) power value for a representation of the EEG signal differs to another, given a set of data samples from a defined time period. Such a “mean difference” test is performed by the processing system 109 for every signal representation.

Preferably, the processing system 109 implements an Analysis of Variance (ANOVA) F ratio test to search for differences in activation, combined with a paired Student's T test. The T test is functionally equivalent to the one way ANOVA test for two groups, but also allows for a measure of direction of mean difference to be analysed (i.e. whether the mean value of a mental state 1 is larger than the mean value for a mental state 2, or vice versa). The general formula for the Student's T test is:

$$t = \frac{mean of mental state 1 - mean of mental state 2}{\sqrt{( \frac{variance of mental state 1}{n for mental state 1} ) + ( \frac{variance of mental state 2}{n for mental state 2} )}}$$

The “n” which makes the denominator in the lower half of the T equation is the number of time series recorded for a particular mental state which make up the means being contrasted in the numerator. (i.e. the number of overlapping or non-overlapping epochs recorded during an update.

The subsequent t value is used in a variety of ways by the processing system 109, including the rating of the feature space dimensions to determine the significance level of the many thousands of features that are typically analysed. Features may be weighted on a linear or non-linear scale, or in a binary fashion by removing those features which do not meet a certain level of significance.

The range of t values that will be generated from the many thousands of hypothesis tests during a signature update can be used to give an overall indication to the user of how far separated the detected mental states are during that update. The t value is an indication of that particular mean separation for the two actions, and the range of t values across all coordinates provides a metric for how well, on average, all of the coordinates separate.

The above-mentioned techniques are termed univariate approaches as the processing system 109 performs the analysis for each individual coordinate at a time, and make feature selections decisions based on those individual t test or ANOVA test results. Corrections may be made at step 406 to adjust for the increased chance of probability error due to the use of the mass univariate approach. Statistical techniques suitable for this purpose include the following multiplicity correction methods: Bonferroni, False Discovery Rate and Dunn Sidack.

An alterative approach is for the processing system 109 to analyse all coordinates together in a mass multivariate hypothesis test, which would account for any potential covariation between coordinates. The processing system 109 can therefore employ such techniques as Discriminant Function Analysis and Multivariate analysis of variance (MANOVA), which not only provides a means to select feature space in a multivariate manner, but also allows the use of eigenvalues created during the analysis to actually classify unknown signal representations in a real-time environment.

At step 408, the processing system 109 prepares for classifying incoming real-time data by weighting the coordinates so that those with the greatest significance in detecting a particular mental state are given precedence. This can be carried out by applying adaptive weight preparation, neural network training or statistical weightings.

The signatures stored in the memory of the processing system 109 are updated or calibrated at step 410. The updating process involves taking data samples, which is added to the evolving database. This data is elicited for the detection of a particular mental state. For example, to update a willed effort mental state, a user is prompted to focus on that willed effort and signal data samples are added to the database and used by the processing system 109 to modify the signature for that detection. When a signature exists, detections can provide feedback for updating the signatures that define that detection. For example, if a user wants to improve their signature for willing an object to be pushed away, the existing detection can be used to provide feedback as the signature is updated. In that scenario, the user sees the detection improving, which provides reinforcement to the updating process.

At step 412, a supervised learning algorithm dynamically takes the update data from step 410 and combines it with the evolving database of recorded data samples to improve the signatures for the mental state that has been updated. Signatures may initially be empty or be prepared using historical data from other users which may have been combined to form a reference or universal starting signature.

At step 414, the signature for the mental state that has been update is made available for mental state classification (at step 400) as well as signature feedback rating at step 416. As a user develops a signature for a given mental state, a rating is available in real-time which reflects how the mental state detection is progressing. The apparatus 100 can therefore provide feedback to a user to enable them to observe the evolution of a signature over time. The discussion above has focused on determination of the presence or absence of a particular mental state. However, it is also possible to determine the intensity of that particular mental state. The intensity can be determined by measuring the “distance” of the transformed signal from the user to a signature. The greater the distance, the lower the intensity. To calibrate the distance to the subjective intensity experienced by the user to an intensity scale, the user can queried regarding the strength of the mental state. The resulting feedback from the user is applied to adjust the weights to calibrate the distance to the intensity scale.

It will be appreciated from the foregoing that the apparatus 100 advantageously enables the online creation of signatures in near real-time. The detection of a user's mental state and creation of a signature can be achieved in a few minutes, and then refined over time as the user's signature for that mental state is updated. This can be very important in interactive applications, where a short term result is important as well as incremental improvement over time.

It will also be appreciated from the foregoing that the apparatus 100 advantageously enables the detection of a mental state having a pregenerated signature (whether predefined or created for the particular user) in real-time. Thus, the detection of the presence or absence of a user's particular mental state, or the intensity of that particular mental state, can be achieved in real-time.

Moreover, signatures can be created for mental states that need not be predefined. The apparatus 100 can classify mental states that are recorded for, not just mental states that are predefined and elicited via pre-defined stimuli.

Each and every human brain is subtly different. While macroscopic structures such as the main gyri (ridges) and sulci (depressions) are common, it is only at the largest scale of morphology at which these generalizations can be made. The intricately detailed folding of the cortex is as individual as fingerprints. This variation in folding causes different parts of the brain to be near the skull in different individuals.

For this reason the electrical impulses, when measured in combination on the scalp, differ between individuals. This means that the EEG recorded on the scalp must be interpreted differently from person to person. Historically, systems that aim to provide an individual with a means of control via EEG measurement have required extensive training, often of the system used and always by the user.

The mental state detection system described herein can utilize a huge number of feature dimensions which cover many spatial areas, frequency ranges and other dimensions. In creating and updating a signature, the system ranks features by their ability to distinguish a particular mental state, thus highlighting those features that are better able to capture the brain's activity in a given mental state. The features selected by the user reflect characteristics of the electrical signals measured on the scalp that are able to distinguish a particular mental state, and therefore reflect how the signals in their particular cortex are manifested on the scalp. In short, the user's individual electrical signals that indicate a particular mental state have been identified and stored in a signature. This permits real-time mental state detection or generation within minutes, through algorithms which compensate for the individuality of EEG.

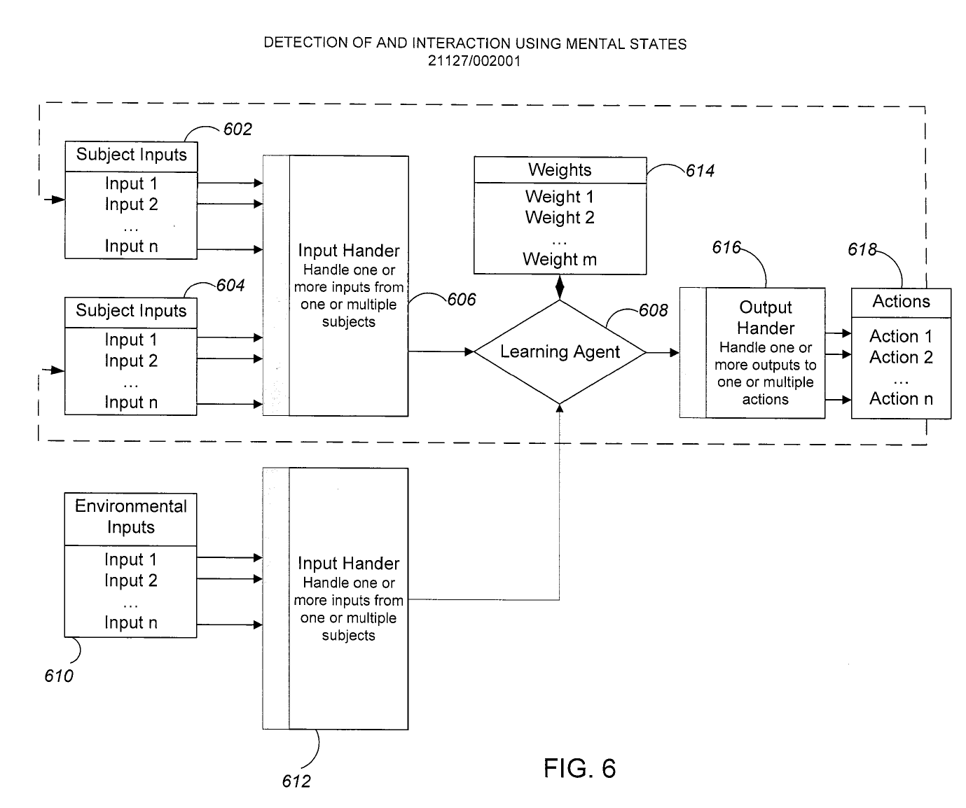

Turning now to the system 30, FIG. 6 shows a schematic representation of a platform 600, which is an embodiment of a system that uses the signals representing mental states. The platform 600 can be implemented as software, as hardware (e.g., an ASIC), or as a combination of hardware and software. The platform is adapted to receive input signals representative of predetermined non-deliberative mental states, e.g., different emotional responses, from one or more subjects. In FIG. 6, input signals representative of an emotional response from a first user are referenced as Input 1 to Input n and are received at a first input device 602, whereas corresponding input signals representative of an emotional response from a second user are received handled by a second input device 604. An input handler 606 handles multiple inputs representative of emotional responses from one or multiple subjects, and facilitates the handling of each input for a neural network or other learning agent 608. Moreover, the platform 600 is adapted to receive a series of environmental inputs from a further device 610, e.g., a sensor or a memory. These environmental inputs are representative of the current state or value of environmental variables that impact in some way one or more subjects. The environmental variables may occur in either a physical environment, such as the temperature or lighting condition in a room, or in a virtual environment, such as the nature of the interaction between a subject and an avatar in an electronic entertainment environment. An input handler 612 acts to process the inputs representative of the environmental variables perceived by the subject, and facilitate the handling of the environmental inputs by the learning agent 608.

A series of weightings 614 are maintained by the platform 600 and used by the learning agent 608 in the processing of the subject and environmental inputs provided by the input handlers 606 and 612. An output handier 616 handles one or multiple output signals provided by the learning agent 608 to an output device 618 adapted to cause multiple possible actions to be carried out that alter selected environmental variables able to be perceived by the subjects.

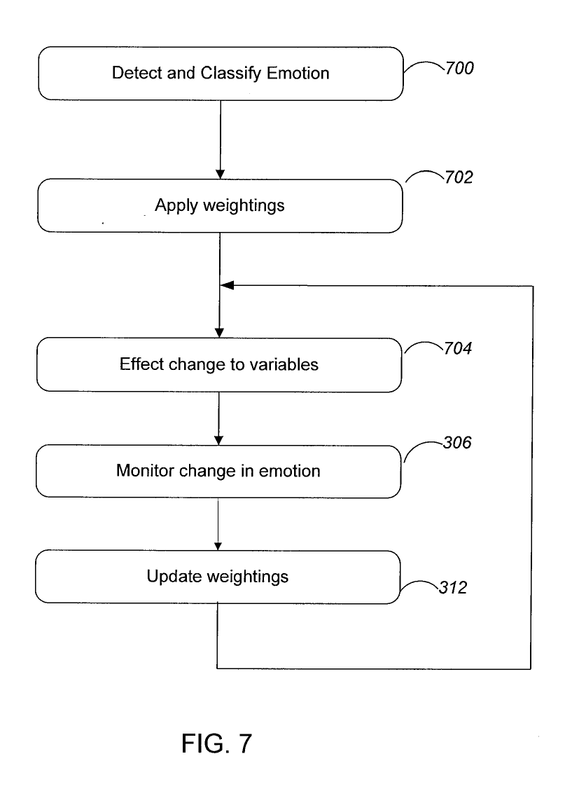

As illustrated in FIG. 7, at step 700, a predetermined non-deliberative mental state, e.g., an emotional response, of one or more of the subjects to which a headset 102 has been fitted is detected and classified. The detected emotional response may be happiness, fear, sadness or any other non-consciously selected emotional response.

The weightings 614 maintained in the platform 600 are each representative of the effectiveness of an environmental variable in evoking a particular emotion in a subject, and are used by the learning agent 608 to select which actions 618 are to be performed in order to bring the emotional response of a user toward a particular emotion, and also to determine the relative change in selected environmental variables that is to be brought about by each of the selected actions.

As each subject interacts with the particular interactive environment in question, the weights are updated by the learning agent 608 in line with the emotional responsiveness of each subject to the change in environmental variables brought about by each of the actions 618.

Accordingly, at step 702, the weightings 604 are applied by the learning agent 408 to the possible actions 418 that can be applied to the environmental variables able to be altered in the interactive environment to cause actions to be performed that are most likely to be effective in evoking a target emotional response in a subject. For example, a particular application may be have a goal of removing an emotional response of sadness. Therefore, for a particular subject, weightings are applied to selection actions, such as causing music to be played and increasing the lighting levels in the room in which the subject, that are likely to evoke an emotional response of happiness, calmness, peace or like positive emotion.

At step 704, the learning agent 608 and output handler 616 cause selected actions 618 to be enacted to thus effect a change in the environmental variables perceived by a subject. At step 706, the emotional response of the user is again monitored by detecting and classifying the presence of an emotional response in the EEG signals of each subject, and the receipt of input signals 602 and 404 representative of the detected emotions at the platform 600. The learning agent 608 observes the relative change in the emotional state of each subject and, at step 708, updates the weightings depending upon their effectiveness; in optimizing the emotional response of the subject.

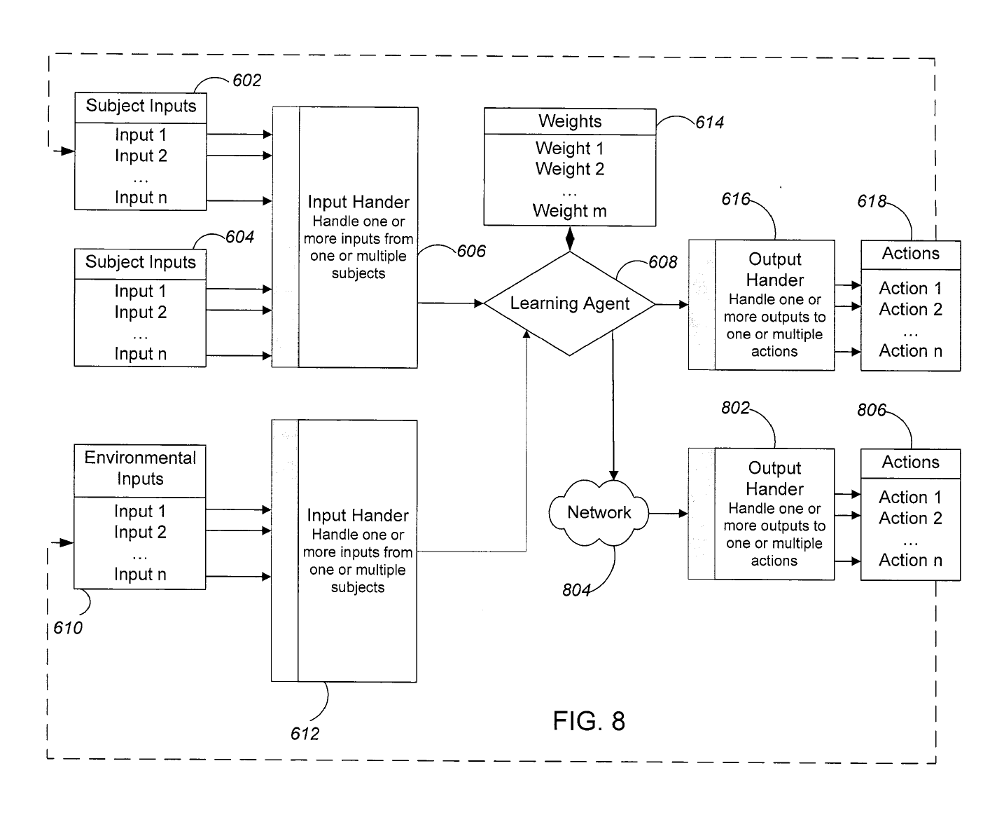

In the example illustrated in FIG. 6, the platform 600 operates in a local interactive environment. FIG. 8 shows an alternate platform 800 operating in both a remote and a networked environment. In addition to processing corresponding detected emotional responses of one or more subjects and states or values of the environmental variable and applying weightings to actions in order to alter selected environmental variables in a local interactive environment, the learning agent 608 is interconnected to a remote output handler 802 via a data network 804, such as the Internet, in order that actions 806 can be performed to alter selected environmental variables perceived by one or more of the subjects. For example, in a gaming environment, the actions 618 may be carried out in a local interactive environment such as a user's local gaming console or personal computer, whereas the actions 806 may be carried out at a remote gaming console or personal computer. In a scenario involving networked gaming consoles, where a first subject is experiencing the emotion of frustration, the learning agent 608 may cause actions to be carried out at a remote gaming console used by another subject in order to alter predetermined parameters at that remote gaming console likely to reduce the level of frustration experienced by the local subject.

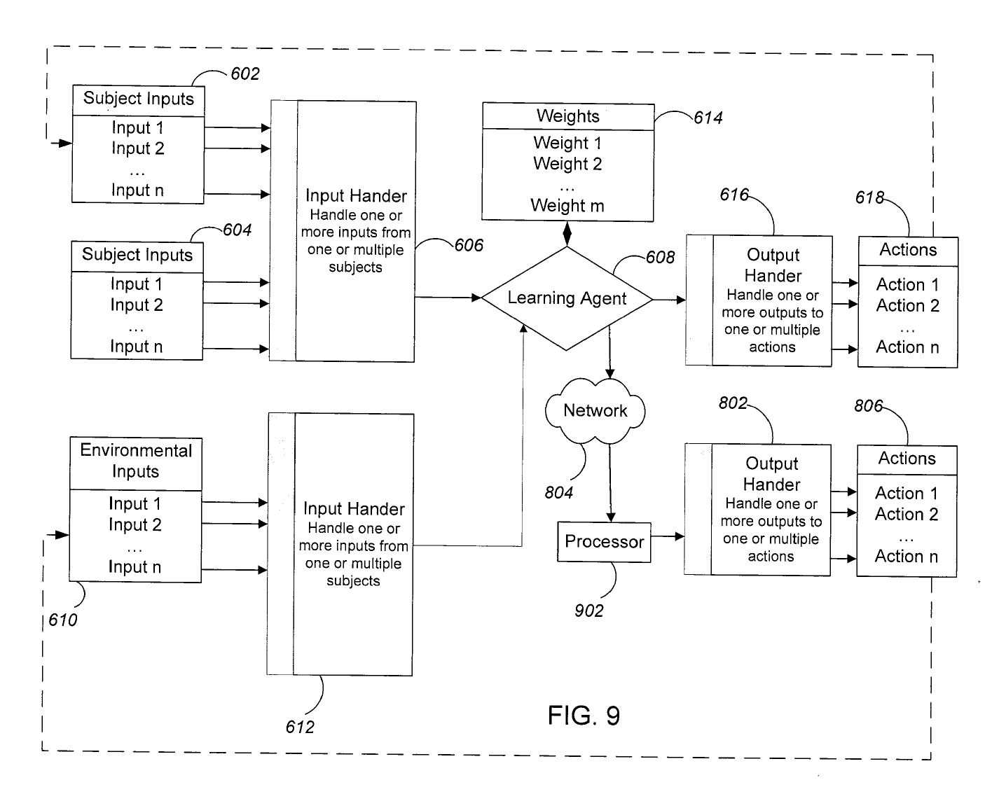

Yet another variant is shown in FIG. 9. The platform 790 shown in that figure is identical to the platform 800 in FIG. 6, with the exception that an extra learning agent or processor 902 is provided between the network 804 and the output handler 802 so that a networked or remote interactive environment is not subject to the alteration of one or more environmental variables by the learning agent 608, but is provided with some local intelligence to take into account local environmental conditions and/or conflicting inputs from one or more other interactive environments with which the processor 902 may be interconnected.

Embodiments of the invention and all of the functional operations described in this specification can be implemented in digital electronic circuitry, or in computer software, firmware, or hardware, including the structural means disclosed in this specification and structural equivalents thereof, or in combinations of them. Embodiments of the invention can be implemented as one or more computer program products, i.e., one or more computer programs tangibly embodied in an information carrier, e.g., in a machine readable storage device or in a propagated signal, for execution by, or to control the operation of, data processing apparatus, e.g., a programmable processor, a computer, or multiple processors or computers. A computer program (also known as a program, software, software application, or code) can be written in any form of programming language, including compiled or interpreted languages, and it can be deployed in any form, including as a stand alone program or as a module, component, subroutine, or other unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub programs, or portions of code). A computer program can be deployed to be executed on one computer or on multiple computers at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

A number of embodiments of the invention have been described. Nevertheless, it will be understood that various modifications may be made without departing from the spirit and scope of the invention.

For example, the invention has been described in the context of queries through the interface to “pull” information from the mental state detection engine 114, but the mental state detection engine can also be configured to “push” information through the interface to the system 30.

As another example, the system 10 can optionally include additional sensors capable of direct measurement of other physiological processes of the subject, such as heart rate, blood pressure, respiration and electrical resistance (galvanic skin response or GSR). Some such sensors, such sensors to measure galvanic skin response, could be incorporated into the headset 102 itself. Data from such additional sensors could be used to validate or calibrate the detection of non-deliberative states.

Accordingly, other embodiments are within the scope of the following claims.

What is claimed is:

1. A method of detecting a mental state, comprising:
receiving, in a processor, bio-signals of a subject from one or more bio-signal detectors; and
determining in the processor whether the bio-signals represent the presence of a particular mental state in the subject.

2. The method of claim 1, wherein the particular mental state comprises a non-deliberative mental state.

3. The method of claim 2, wherein the non-deliberative mental state is an emotion, preference, sensation, physiological state, or condition.

4. The method of claim 1, further comprising generating a signal from the processor representing whether the particular mental state is present.

5. The method of claim 1, wherein the bio-signals comprise electroencephalograph (EEG) signals.

6. The method of claim 1, wherein determining includes transforming the bio-signals into a different representation.

7. The method of claim 6, wherein determining includes calculating values for one or more features of the different representation.

8. The method of claim 7, wherein determining includes comparing the values to a mental state signature.

9. The method of claim 8, wherein the particular mental state comprises a non-deliberative mental state and determining the presence of the non-deliberative mental state is performed substantially without calibration of the mental state signature.

10. The method of claim 1, wherein receiving and determining occur in substantially real time.

11. A computer program product, tangibly stored on machine readable medium, the product comprising instructions operable to cause a processor to:
receive bio-signals from one or more bio-signal detectors; and
determine whether the bio-signals indicate the presence of a particular mental state in a subject.

12. The product of claim 11, wherein the particular mental state comprises a non-deliberative mental state.

13. The product of claim 12, wherein the non-deliberative mental state is an emotion, preference, sensation, physiological state, or condition.

14. The product of claim 11, further comprising instructions operable to cause the processor to generate a signal representing whether the particular mental state is present.

15. The product of claim 11, wherein the bio-signals comprise electroencephalograph (EEG) signals.

16. A system, comprising
a processor configured to receive bio-signals from one or more bio-signal detectors and determine whether the bio-signals indicate the presence of a particular mental state in a subject.

17. The system of claim 16, wherein the particular mental state comprises a non-deliberative mental state.

18. The system of claim 17, wherein the non-deliberative mental state is an emotion, preference, sensation, physiological state, or condition.

19. The system of claim 16, wherein the processor is configured to generate a signal representing whether the particular mental state is present.

20. The system of claim 16, wherein the bio-signals comprise electroencephalograph (EEG) signals.

21. A method of using a detected mental state, comprising:
receiving, in a processor, a signal representing whether a mental state is present in a subject.

22. The method of claim 21, wherein the particular mental state comprises a non-deliberative mental state.

23. The method of claim 22, wherein the non-deliberative mental state is an emotion, preference, sensation, physiological state, or condition.

24. The method of claim 21, further comprising storing the signal.

25. The method of claim 21, further comprising selecting an action to modify an environment based on the signal.

26. The method of claim 21, wherein the non-deliberative state is an emotion, and the method comprises:
storing data representing a target emotion;
determining with the processor an alteration to an environmental variable that is expected to alter an emotional response of a subject toward the target emotion; and
causing the alteration of the environmental variable.

27. The method of claim 26, further comprising determining whether the target emotion has been evoked based on signals representing whether the emotion is present in the subject.

28. The method of claim 27, further comprising storing weightings representing an effectiveness of the environmental variable in evoking the target emotion and using the weightings in determining the alteration.

29. The method of claim 28, further comprising updating the weightings with a learning agent based on the signals representing whether the emotion is present.

30. The method of claim 19, wherein the environmental variables occur in a physical or virtual environment.

31. A computer program product, tangibly stored on machine readable medium, the product comprising instructions operable to cause a processor to:
receive at a processor a signal representing whether a mental state is present in a subject.

32. The product of claim 31, wherein the particular mental state comprises a non-deliberative mental state.

33. The product of claim 32, wherein the non-deliberative mental state is an emotion, preference, sensation, physiological state, or condition.

34. The product of claim 31, further comprising instructions to cause the processor to store the signal.

35. The method of claim 31, further comprising instructions to cause the processor to modify an environment based on the signal.

36. A system, comprising
a processor configured to receive a signal representing whether a mental state is present in a subject.

37. The system of claim 36, wherein the particular mental state comprises a non-deliberative mental state.

38. The system of claim 37, wherein the non-deliberative mental state is an emotion, preference, sensation, physiological state, or condition.

39. The system of claim 36, further comprising instructions to cause the processor to store the signal.

40. The method of claim 36, further comprising instructions to cause the processor to modify an environment based on the signal.

41. A method of detecting and using a mental state, comprising:
detecting bio-signals of a subject with one or more bio-signal detectors;
directing the bio-signals to a first processor;
determining in the first processor whether the bio-signals represent the presence of a particular mental state in the subject;
generating a signal from the first processor representing whether the particular mental state is present;
receiving the signal at a second processor; and
storing the signal or modifying an environment based on the signal.

42. An apparatus comprising:
one or more bio-signal detectors;
a first processor configured to bio-signals from the one or more bio-signal detectors, determine whether the bio-signals indicate the presence of a particular mental state in a subject, and generate a signal representing whether the particular mental state is present;
a second processor configured to receive the signal and store the signal or modify an environment based on the signal.

43. A method of interaction of a user with an environment, comprising:
detecting and classifying the presence of a predetermined mental state in response to one or more biosignals from the user;
selecting one or more environmental variables that affect an emotional response of the user; and
performing one or more actions to alter the selected environmental variables and thereby alter the emotional response of a user.
